(19) United States
(12) Patent Application Publication
(10) Pub. No.: US 2024/0166997 A1
(43) Pub. Date: May 23, 2024

(54) CELL CULTURE SURFACES AND CONTAINERS AND METHODS FOR MAKING AND USING THEM

(71) Applicant: SAINT-GOBAIN PERFORMANCE PLASTICS CORPORATION, Solon, OH (US)

(72) Inventors: Pierre-Luc Girard-Lauriault, Montreal (CA); Gad Sabbatier, Montreal (CA); Balaji Ramachandran, Montreal (CA)

(21) Appl. No.: 18/515,546

(22) Filed: Nov. 21, 2023

Related U.S. Application Data
(60) Provisional application No. 63/384,466, filed on Nov. 21, 2022.

(51) Int. Cl. C12N 5/0784; C12M 1/12

(52) U.S. Cl. CPC C12N 5/0639; C12M 25/04; C12N 2501/22; C12N 2533/20; C12N 2537/00

(57) ABSTRACT

The disclosure relates to coated surfaces suitable for cell culture, to methods for making such surfaces, and to methods for culturing adherent cells on such surfaces. One aspect of the disclosure is a surface suitable for cell culture including an amorphous hydrogenated carbon coating. Another aspect of the disclosure is a method for making a surface suitable for cell culture including an amorphous hydrogenated carbon coating. Another aspect of the disclosure is a method for culturing adherent cells that includes incubating a surface including an amorphous hydrogenated carbon coating with adherent cells and a growth medium.

### CROSS-REFERENCE TO RELATED APPLICATIONS

This application claims the benefit of priority of U.S. Provisional Patent Application No. 63/384,466, filed Nov. 21, 2022, which is hereby incorporated herein by reference in its entirety.

### BACKGROUND OF THE DISCLOSURE

### Field of the Disclosure

The present disclosure relates generally to coatings for cell culture devices. More particularly, the disclosure relates to coated surfaces suitable for cell culture, to methods for making such surfaces, and to methods for culturing adherent cells on such surfaces.

### Technical Background

In vitro cell culture is a complex process in which cells are grown under controlled conditions, generally outside of a natural environment but under conditions that are similar enough to in vivo conditions to allow for growth, and, in the case of adherent cells, adhesion to a growth surface. It is typically cells derived from multi-cellular eukaryotes, especially animal cells that are cultured. However, cells from plants, fungi, and insects and microbes including viruses, bacteria and protista can also be cultured.

In vitro cell culture can provide materials not only for research but also for various applications in pharmacology, physiology, and toxicology. Cell culture can also be desirable for use in bioprocessing and cell therapy.

In typical cell culture processes, cells can be grown and maintained at an appropriate temperature and gas mixture in a cell incubator. Typically, mammalian cells are incubated at 37° C. with a pH maintained between 7.2 and 7.4. The pH can be controlled using a bicarbonate buffering system in the medium in conjunction with an incubator atmosphere of approximately 5-7 vol % carbon dioxide. Carbon dioxide reacts with water to form carbonic acid, which in turn interacts with bicarbonate ions in the medium to form a buffering system that can maintain the pH near physiological levels. Oxygen is essential for cellular metabolism and growth for many desirable cell types. Culture conditions can vary for each cell type, and variation of conditions for a particular cell type can result in the expression of different phenotypes. For instance, bicarbonate-based buffers can be substituted with mono- and di- or tri-sodium phosphate buffers, chloride and ammonia buffers, lactate, or organic buffers such as HEPES, etc.

Commercially-available cell culture containers in the form of cell culture bags are a conventional format used for cell culture. Cell culture bags have the advantage of being disposable, which reduces preparation and clean-up time. Additionally, cell culture bags are pre-sterilizable, inexpensive, easy to use and require minimal space for storage and use. Disposables also help reduce the risk of contamination for the cell culture and for the environment.

Cell culture bags are often provided with a fluoropolymer inner surface. Fluoropolymer surfaces are highly advantageous because they can be provided with a very low amount of leachable organic material, which can be important in many cell culture applications. Fluoropolymer surfaces also resist adsorption of proteins. Fluoropolymer surfaces are further advantaged from the standpoint of manufacturability and sterilizability.

However, fluoropolymer surfaces have generally low surface energy, and as such, it can be difficult for adherent cells to adhere to such surfaces. This can make cell culture systems (e.g., cell culture devices) based on fluoropolymer surfaces much less useful for culturing adherent cells.

Accordingly, there remains a need for a surface suitable for cell culture, especially for adherent cells.

### SUMMARY OF THE DISCLOSURE

Accordingly, one aspect of the disclosure is a surface suitable for cell culture. The surface includes
a substrate having a fluoropolymer surface; and
an amorphous hydrogenated carbon coating disposed on the fluoropolymer surface of the substrate, the amorphous hydrogenated carbon coating having a first zone of thickness proximal to and extending from the fluoropolymer surface and a second zone of thickness distal to the fluoropolymer surface and at a surface of the amorphous hydrogenated carbon coating, the amorphous hydrogenated carbon coating having a higher total concentration of oxygen and nitrogen in the second zone of thickness than in the first zone of thickness.

Another aspect of the disclosure is a method for making a surface suitable for cell culture, e.g., as otherwise described herein. The method includes
depositing on a fluoropolymer surface of a substrate of the surface an amorphous hydrogenated carbon coating, the deposition including:
   depositing via chemical vapor deposition (e.g., plasma-enhanced chemical vapor deposition (PECVD)) a first thickness of the amorphous hydrogenated carbon coating in a first zone thereof using a combination of a hydrocarbon gas (e.g., ethylene or propylene) and CO₂ at a first ratio of hydrocarbon gas to CO₂, wherein the first zone is proximal to and extending from the fluoropolymer surface; and
   depositing via chemical vapor deposition (e.g., PECVD) a second thickness of the amorphous hydrogenated carbon coating in a second zone thereof using a combination of a hydrocarbon gas (e.g., ethylene or propylene) and at least one of CO₂ and NH₃, wherein the second zone is distal to the fluoropolymer surface and at a surface of the amorphous hydrogenated carbon coating, and wherein the amorphous hydrogenated carbon coating has a higher total concentration of oxygen and nitrogen in the second zone than in the first zone thereof.

Another aspect of the disclosure is a method for culturing adherent cells that includes incubating a surface as described herein with adherent cells and a growth medium.

In various embodiments of the surfaces and methods described herein, the fluoropolymer surface is an activated fluoropolymer surface. For example, in some embodiments, the fluoropolymer surface is activated by a plasma treatment (e.g., with an ammonia plasma or CO₂ plasma), or by a corona treatment such as a C-treatment.

Other aspects of the disclosure will be apparent based on the description provided herein.

### BRIEF DESCRIPTION OF THE DRAWINGS

FIG. 1 is a schematic, cross-sectional view of a surface suitable for cell culture applications according to one embodiment of the present disclosure.

FIG. 2 is a schematic, cross-sectional view of a surface suitable for cell culture applications according to another embodiment of the present disclosure.

FIG. 3 provides a schematic plan view and a schematic cross-sectional view of a cell culture container of the disclosure.

FIG. 4 is a diagram showing an elemental percentage of oxygen and of nitrogen on the surface described in Examples 1-8.

FIG. 5 is a diagram showing a water contact angle of the surface described in Examples 1-8.

FIG. 6 is a diagram showing an exemplary thickness of a coating disposed on the surface described in Examples 3-8.

FIG. 7 is a diagram showing cell adhesion and proliferation of hMSCs on the coated surface described in Examples 5 and 8, in comparison to that on an un-activated fluoropolymer surface, an activated fluoropolymer surface, and commercially available TCPS surfaces.

FIG. 8 is a diagram showing cell adhesion and proliferation of monocyte-derived dendritic cells on the coated surface described in Examples 5 and 8, respectively, in comparison to that on an un-activated fluoropolymer surface, an activated fluoropolymer surface, and a commercially available TCPS surface.

### DETAILED DESCRIPTION

The present inventors have noted various ways that can be used to treat a fluoropolymer surface in an effort to provide improved adhesion for adherent cells. Treatment of a fluoropolymer surface with a plasma treatment may increase hydrophilicity (e.g., as measured by a surface contact angle), which might promote cell adhesion and proliferation. However, simply increasing the hydrophilicity of a surface of a cell culture device may not be sufficient to provide a desirable surface for culturing adherent cells.

Cell culture devices may be coated with pro-adherent proteins such as laminin, fibronectin or collagen. These proteins are generally obtained from animals or humans, and must be purified and characterized for clinical applications. However, the purification and characterization process can be expensive and time-consuming, which ultimately increases the cost of the cell culture devices.

The present inventors note that cell culture surfaces having a relatively high concentration of nitrogenous and/or oxygenous functionality (e.g., amine and/or carboxylic acid functional groups) at surfaces thereof may help enhance cell adhesion and proliferation. For example, cell culture devices having amine and/or carboxylic acid functional groups may generally exhibit better cell culture kinetics in a serum-free cell culture than those without such functional groups. However, since amine and carboxylic acid can react with oxygen and also provoke surface reorganization, the amount of these functional groups on the surfaces may decrease over time and thus reduce surface functionalities of the cell culture devices.

The present inventors have determined that desirable cell culture systems for adherent cell culture can be provided by coating fluoropolymer layers at a surface thereof with a coating that presents nitrogenous and/or oxygenous functionality. Plasma polymerization can be used to provide such a coating in the form of an amorphous coating. However, the present inventors have determined that if a homogeneous coating is provided with a desirably high degree of nitrogenous and/or oxygenous functionality at the surface, such coating can be unstable during the autoclaving procedures typically used to sterilize surfaces for cell culture. The present inventors have determined that a coating with a lower amount of nitrogenous and/or oxygenous functionality proximal to the fluoropolymer surface and a higher amount of nitrogenous and/or oxygenous functionality distal to the fluoropolymer surface and at a surface of the coating, e.g., a non-homogeneous coating, can provide a system that not only has desirable properties at the coating surface for cell culture, but also provides a tenacious enough coating to survive autoclaving. The present inventors have noted that the coatings described herein can be functionalized to provide especially suitable surfaces for adherent cell culture, while being sterilizable (e.g., by autoclaving), protein-free, and relatively straightforward and inexpensive to fabricate.

Accordingly, one aspect of the disclosure is a surface suitable for cell culture applications. The surface may be, for example, a surface of a cell culture device (e.g., a cell culture bag or a cell culture tube). In various embodiments, the surface may be an inner surface of the cell culture device. The surface includes a substrate having a fluoropolymer surface. The surface also includes an amorphous hydrogenated carbon coating disposed on the fluoropolymer surface of the substrate. The amorphous hydrogenated carbon coating has a first zone of thickness proximal to and extending from the fluoropolymer surface, and a second zone of thickness distal to the fluoropolymer surface and at a surface of the amorphous hydrogenated carbon coating. Notably, the amorphous hydrogenated carbon coating has a higher total concentration of oxygen and nitrogen in the second zone of thickness than in the first zone of thickness. As used herein, the term “concentration” means an elemental percentage based on atomic weight.

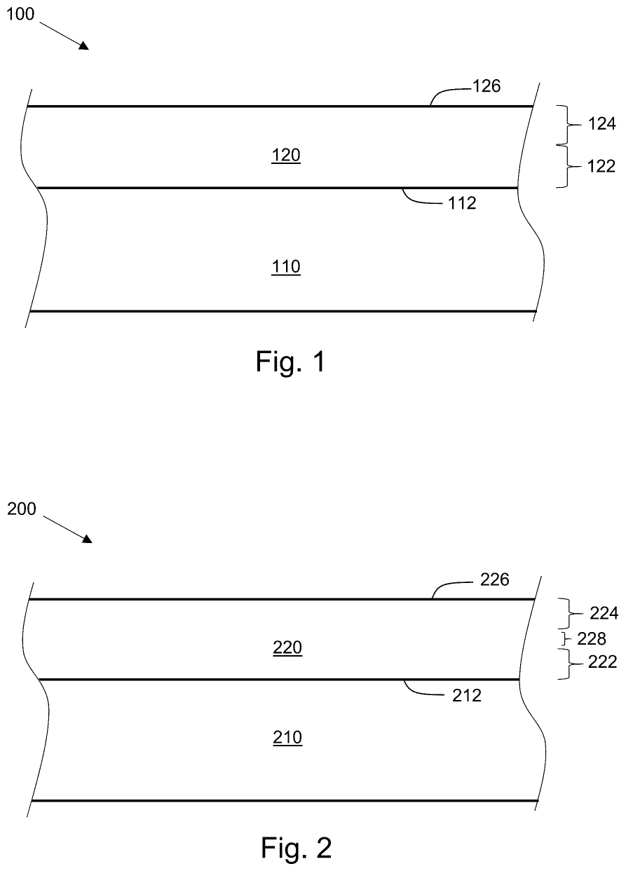

One embodiment of a surface according to the disclosure is shown in a schematic, cross-sectional view in FIG. 1. In this embodiment, a surface 100 for cell culture applications includes a substrate 110 having a fluoropolymer surface 112. The surface 100 further includes an amorphous hydrogenated carbon coating 120 disposed on the fluoropolymer surface 112 of the substrate 110. The amorphous hydrogenated carbon coating 120 has a first zone of thickness 122 proximal to and extending from the fluoropolymer surface 112, and a second zone of thickness 124 distal to the fluoropolymer surface 112 and at a surface 126 of the amorphous hydrogenated carbon coating 120. The amorphous hydrogenated carbon coating 120 has a higher total concentration of oxygen and nitrogen in the second zone of thickness 124 than in the first zone of thickness 122. Notably, the embodiment of FIG. 1 shows that the first zone of thickness 122 is contiguous with the second zone of thickness 124.

A variety of fluoropolymers can be used to provide the fluoropolymer surface. In various desirable embodiments as otherwise described herein, the fluoropolymer surface is a fluorinated ethylene propylene (FEP) surface. Other fluoropolymers can alternatively be used. For example, in various embodiments as otherwise described herein, the fluoropolymer surface is a polytetrafluoroethylene (PTFE) surface, a perfluoroalkoxy (PFA) surface, an ethylene tetrafluroethylene (ETFE) surface, a polyvinylidene fluoride (PVDF) surface, a polychlorotrifluoroethylene (PCTFE) surface, an ethylene chlorotrifluoroethylene (ECTFE) surface, an ethylene fluorinated ethylene propylene (EFEP) surface, a perfluoropolyether (PFPE) surface, a modified polytetrafluoroethylene (TFM) surface, a polyvinyl fluoride surface, or a combination of any two or more thereof.

In various desirable embodiments as otherwise described herein, the fluoropolymer surface is a surface of a fluoropolymer-silicone laminate film. The present inventors note that silicone-fluoropolymer laminates can provide a good balance of physical properties, high oxygen- and carbon dioxide (CO₂)-permeability, and low leachability of organics. The silicone-fluoropolymer can be, for example, a silicone-FEP laminate providing an FEP fluoropolymer surface. Silicone-fluoropolymer laminates are described in U.S. Pat. No. 9,926,524, which is hereby incorporated herein by reference in its entirety. Cell culture bags based on silicone-fluoropolymer laminates are commercially available under the trade name VueLife®, from Saint-Gobain Performance Polymer Products Corporation.

As the person of ordinary skill in the art would appreciate, to allow for a cell culture medium to absorb oxygen from the atmosphere and desorb CO₂ to the atmosphere through a surface of the cell culture, it can be desirable for cell culture systems to have a degree of oxygen and CO₂ permeability.

Accordingly, in various desirable embodiments as otherwise described herein, the fluoropolymer surface as described herein is a surface of a film having an oxygen permeability of at least 1500 cc/m²-day-atm, e.g., at least 1800 cc/m²-day-atm, or in the range of 1500-20000 cc/m²-day-atm, or 1800-16000 cc/m²-day-atm. Oxygen permeability is measured using a MOCON OxTran 220 O₂TR Analyzer, following ASTM D3985 with test conditions: temperature: 23° C.; test gas: 10% O₂ in N₂; humidity: 0% relative humidity on both sides of the film; carrier gas flow rate: 20 sccm N₂; test area: 5 cm₂; exam cycle: 15 minutes.

In various desirable embodiments as otherwise described herein, the fluoropolymer surface as described herein is a surface of a film having a CO₂ permeability of at least 3500 cc/m²-day-atm, e.g., 4000 cc/m²-day-atm, or in the range of 3500-25000 cc/m²-day-atm, 4000-23000 cc/m²-day-atm. CO₂ permeability is measured using a MOCON Permatran-C 441 CO₂TR Analyzer, following ASTM F2476 with test conditions: temperature: 23° C.; test gas: 100% CO₂; humidity: 0% relative humidity on both sides of the film; carrier gas flow rate: 50 sccm N₂; test area: 5 cm₂; exam cycle: 30 minutes.

In various desirable embodiments as otherwise described herein, the fluoropolymer surface as described herein is a surface of a film having a water vapor transmission rate (WVTR) in the range of 0.65-1 g/m²-day-atm, e.g., in the range of 0.72-0.94 g/m²-day-atm. WVTR is measured using a MOCON Permatran W700 Water Vapor Analyzer following ASTM F1249 with test conditions: temperature: 23° C.; humidity: 100% RH on test gas side; 0% RH on detector side of film; carrier gas flow rate: 10 sccm N₂; test area: 50 cm²; exam cycle: 60 minutes.

The present inventors have noted that adhesion of the amorphous hydrogenated carbon coating to the fluoropolymer surface can be improved by using an activated fluoropolymer surface, e.g., a treated fluoropolymer surface. Accordingly, in various desirable embodiments as otherwise described herein, the fluoropolymer surface is an activated fluoropolymer surface. An activated fluoropolymer surface provides oxygenous and/or nitrogenous functionality. Without intending to be bound by theory, the present inventors surmise that such functionality provides a reactive surface from which an amorphous film can be grown, for example, by plasma polymerization. The amorphous film can be covalently bound to the activated fluoropolymer surface.

A variety of processing techniques can be used to provide the activated fluoropolymer surface. For example, a plasma treatment can be used, such as by treatment with an ammonia (NH₃) plasma, a CO₂ plasma, or a plasma of a combination of NH₃ and CO₂. The present inventors have determined that treatment of the fluoropolymer surface with an NH₃ plasma can better increase the hydrophilicity and biocompatibility of the fluoropolymer surface than treatment with a CO₂ plasma, and thus can better promote cell adhesion and proliferation on the cell culture surface. In other embodiments, the plasma can be an oxygen plasma, a nitrogen plasma, or a plasma of a combination of oxygen and nitrogen. The present inventors surmise, without intending to be bound by theory, that plasma treatments can not only add nitrogenous and/or oxygenous functionalities at the fluoropolymer surface, but also remove some fluorine from the fluoropolymer surface.

In other embodiments, the fluoropolymer surface can be activated (e.g., treated) by a corona treatment, such as a C-treatment. As used herein, the corona treatment, also called corona discharge, is a plasma treatment of a surface in an atmosphere comprising an organic gas. The organic gas can be based on, for example, a ketone or an alcohol. In various embodiments, the alcohol includes four carbon atoms or less. In an embodiment, the organic gas is acetone. In an embodiment, the organic gas is admixed with an inert gas such as nitrogen. The acetone/nitrogen atmosphere causes an increase of adhesion of the fluoropolymer layer to the layer that it directly contacts. In an embodiment, the treatment causes an increase of adhesion of the fluoropolymer layer to the polymeric layer. In an exemplary embodiment, the treatment includes C-treatment of a C-treatable fluoropolymer. C-treatment is further disclosed in U.S. Pat. Nos. 6,726,979 and 8,559,100, each of which is hereby incorporated herein by reference in its entirety.

In various embodiments as otherwise described herein, the activated fluoropolymer surface has a concentration of oxygen less than 5 atom %, and a concentration of nitrogen less than 10 atom %. As used herein, atom % s are determined by x-ray photoelectric spectroscopy, and as such are atomic percents based on all atoms except hydrogen, which the person of ordinary skill in the art appreciates is not generally detectable by XPS.

In various embodiments as otherwise described herein, the activated fluoropolymer surface can desirably have a reduced water contact angle. As the person of ordinary skill in the art will appreciate, a surface that has a higher amount of nitrogenous and/or oxygenous functionality generally has a lower water contact angle. The water contact angle can thus be used to measure the extent of activation of the fluoropolymer surface. In various embodiments as otherwise described herein, the activated fluoropolymer surface has a water contact angle in a range from 60° to 120°.

As noted above, disposed on the fluoropolymer surface (e.g., an activated fluoropolymer surface) of the substrate is an amorphous hydrogenated carbon coating. An amorphous hydrogenated carbon coating, as used herein, is a coating that has significant hydrogen and carbon content, and is substantially non-crystalline in nature. As described in detail below, such coatings can be made by polymerization of a polymerizable hydrocarbon (e.g., ethylene or propylene) under chemical vapor deposition conditions, for example, by plasma enhanced chemical vapor deposition (PECVD).

The person of ordinary skill in the art can, based on the disclosure herein, determine a desirable thickness for the amorphous hydrogenated carbon coating. In various desirable embodiments as otherwise described herein, the amorphous hydrogenated carbon coating has a thickness in the range of 10-200 nm, e.g., 10-100 nm, or 10-75 nm, or 10-50 nm, or 15-200 nm, or 15-100 nm, or 15-75 nm, or 15-50 nm, or 20-200 nm, or 20-100 nm, or 20-75 nm, or 20-50 nm. But other thicknesses can be used.

The amorphous hydrogenated carbon coatings described herein, in various embodiments, have little, if any, graphitic character. In various desirable embodiments as otherwise described herein, the amorphous hydrogenated carbon coating has a ratio of hydrogen to carbon of at least 1, e.g., at least 1.2, or at least 1.4. The ratio of hydrogen to carbon can be measured as described in P.-L. Girard-Lauriault et al., “Chemical Characterisation of Nitrogen-Rich Plasma-Polymer Films Deposited in Dielectric Barrier Discharges at Atmospheric Pressure,” Plasma Process. Polym., 5, 631-44 (2008), which is hereby incorporated herein by reference in its entirety.

As noted above, it can be advantageous to have a coating with a lower amount of nitrogenous and/or oxygenous functionality proximal to the fluoropolymer surface and a higher amount of nitrogenous and/or oxygenous functionality distal to the fluoropolymer surface and at a surface of the coating. This can provide a more highly cross-linked layer at the interface with the fluoropolymer substrate, and as such can provide a stable polymer backbone and thus enhance the stability of the coating. Hence, in the embodiment of FIG. 1, the amorphous hydrogenated carbon coating 120 has a higher concentration of hydrocarbon in the first zone of thickness 122 than in the second zone of thickness 124, and forms a more cross-linked layer in the first zone of thickness 122. In various desirable embodiments as otherwise described herein, the amorphous hydrogenated carbon coating has, in the first zone of thickness, a total concentration of oxygen and nitrogen that is at least 3 atom % less than that a total concentration of oxygen and nitrogen in the second zone of thickness, e.g., at least 4 atom % less, or at least 5 atom % less, or at least 6 atom % less, or at least 7 atom % less. However, in many embodiments, the amorphous hydrogenated carbon coating has, in the first zone of thickness, a total concentration of oxygen and nitrogen of at least 5 atom %, e.g., at least 10 atom %, or at least 15 atom %.

The person of ordinary skill in the art can, based on the disclosure herein, determine a desirable thickness for the first zone of thickness of the amorphous hydrogenated carbon coating. In various desirable embodiments as otherwise described herein, the first zone of thickness of the amorphous hydrogenated carbon coating has a thickness in the range of 8-190 nm, e.g., 8-100 nm, or 8-75 nm, or 8-50 nm, or 15-190 nm, or 15-100 nm, or 15-75 nm, or 15-50 nm, or 20-190 nm, or 20-100 nm, or 20-75 nm, or 20-50 nm. But other thicknesses can be used.

Further, as noted above, it is advantageous to have a coating with a higher amount of nitrogenous and/or oxygenous functionality distal to the fluoropolymer surface and at a surface of the coating. Thus, the coating can provide a more highly-functionalized layer at the surface of the coating. The present inventors have determined that a more functionalized layer of the coating, for example, with the presence of carboxylic acid and/or amine functional groups, can enhance the functionality of the coating, especially with respect to adhesion of adherent cells. Accordingly, in the embodiment of FIG. 1, the amorphous hydrogenated carbon coating 120 includes a higher concentration of oxygen and/or nitrogen in the second zone of thickness 124 than in the first zone of thickness 122, and as such can provide a more functionalized layer in the second zone of thickness 124. In various desirable embodiments as otherwise described herein, the amorphous hydrogenated carbon coating has a total concentration of oxygen and nitrogen in the second zone of thickness of at least 10 atom %, e.g., at least 15 atom %, or at least 20 atom %.

The person of ordinary skill in the art can, based on the disclosure herein, determine a desirable thickness for the second zone of thickness of the amorphous hydrogenated carbon coating. In various desirable embodiments as otherwise described herein, the second zone of thickness of the amorphous hydrogenated carbon coating has a thickness in the range of 2-100 nm, e.g., 2-50 nm, or 2-35 nm, or 2-20 nm, or 5-100 nm, or 5-50 nm, or 5-35 nm, or 5-20 nm, or 5-10 nm, or 10-100 nm, or 10-50 nm, or 10-35 nm, or 10-20 nm, or 20-100 nm, or 20-50 nm. But other thicknesses can be used.

In various desirable embodiments as otherwise described herein, the amorphous hydrogenated carbon coating includes oxygen proximal to the fluoropolymer surface, e.g., in the first zone of thickness. Accordingly, in various embodiments, the amorphous hydrogenated carbon coating in the first zone of thickness thereof is made up of at least 90 atom %, e.g., at least 95 atom % carbon and oxygen (i.e., discounting hydrogen from the analysis as described above). In various such embodiments, the amorphous hydrogenated carbon coating can include oxygen distal to the fluoropolymer surface and at a surface of the coating, e.g., in the second zone of thickness. Accordingly, in various such embodiments, the amorphous hydrogenated carbon coating in the second zone of thickness thereof is made up of at least 90 atom %, e.g., at least 95 atom % carbon and oxygen (i.e., discounting hydrogen from the analysis as described above). In various such embodiments, the amorphous hydrogenated carbon coating can include nitrogen distal to the fluoropolymer surface and at a surface of the coating, e.g., in the second zone of thickness. Accordingly, in various such embodiments, the amorphous hydrogenated carbon coating in the second zone of thickness thereof is made up of at least 90 atom %, e.g., at least 95 atom % carbon and nitrogen (i.e., discounting hydrogen from the analysis as described above). And in various such embodiments, the amorphous hydrogenated carbon coating can include nitrogen and oxygen distal to the fluoropolymer surface and at a surface of the coating, e.g., in the second zone of thickness. Accordingly, in various such embodiments, the amorphous hydrogenated carbon coating in the second zone of thickness thereof is made up of at least 90 atom %, e.g., at least 95 atom % carbon, nitrogen and oxygen (i.e., discounting hydrogen from the analysis as described above).

In various desirable embodiments as otherwise described herein, the amorphous hydrogenated carbon coating includes oxygen and nitrogen proximal to the fluoropolymer surface, e.g., in the first zone of thickness. Accordingly, in various embodiments, the amorphous hydrogenated carbon coating in the first zone of thickness thereof is made up of at least 90 atom %, e.g., at least 95 atom % carbon, nitrogen and oxygen (i.e., discounting hydrogen from the analysis as described above). In various such embodiments, the amorphous hydrogenated carbon coating can include oxygen distal to the fluoropolymer surface and at a surface of the coating, e.g., in the second zone of thickness. Accordingly, in various such embodiments, the amorphous hydrogenated carbon coating in the second zone of thickness thereof is made up of at least 90 atom %, e.g., at least 95 atom % carbon and oxygen (i.e., discounting hydrogen from the analysis as described above). In various such embodiments, the amorphous hydrogenated carbon coating can include nitrogen distal to the fluoropolymer surface and at a surface of the coating, e.g., in the second zone of thickness. Accordingly, in various such embodiments, the amorphous hydrogenated carbon coating in the second zone of thickness thereof is made up of at least 90 atom %, e.g., at least 95 atom % carbon and nitrogen (i.e., discounting hydrogen from the analysis as described above). And in various such embodiments, the amorphous hydrogenated carbon coating can include nitrogen and oxygen distal to the fluoropolymer surface and at a surface of the coating, e.g., in the second zone of thickness. Accordingly, in various such embodiments, the amorphous hydrogenated carbon coating in the second zone of thickness thereof is made up of at least 90 atom %, e.g., at least 95 atom % carbon, nitrogen and oxygen (i.e., discounting hydrogen from the analysis as described above).

In various desirable embodiments as otherwise described herein, the amorphous hydrogenated carbon coating includes nitrogen proximal to the fluoropolymer surface, e.g., in the first zone of thickness. Accordingly, in various embodiments, the amorphous hydrogenated carbon coating in the first zone of thickness thereof is made up of at least 90 atom %, e.g., at least 95 atom % carbon and nitrogen (i.e., discounting hydrogen from the analysis as described above). In various such embodiments, the amorphous hydrogenated carbon coating can include oxygen distal to the fluoropolymer surface and at a surface of the coating, e.g., in the second zone of thickness. Accordingly, in various such embodiments, the amorphous hydrogenated carbon coating in the second zone of thickness thereof is made up of at least 90 atom %, e.g., at least 95 atom % carbon and oxygen (i.e., discounting hydrogen from the analysis as described above). In various such embodiments, the amorphous hydrogenated carbon coating can include nitrogen distal to the fluoropolymer surface and at a surface of the coating, e.g., in the second zone of thickness. Accordingly, in various such embodiments, the amorphous hydrogenated carbon coating in the second zone of thickness thereof is made up of at least 90 atom %, e.g., at least 95 atom % carbon and nitrogen (i.e., discounting hydrogen from the analysis as described above). And in various such embodiments, the amorphous hydrogenated carbon coating can include nitrogen and oxygen distal to the fluoropolymer surface and at a surface of the coating, e.g., in the second zone of thickness. Accordingly, in various such embodiments, the amorphous hydrogenated carbon coating in the second zone of thickness thereof is made up of at least 90 atom %, e.g., at least 95 atom % carbon, nitrogen and oxygen (i.e., discounting hydrogen from the analysis as described above).

In various embodiments, the first zone of thickness of the amorphous hydrogenated carbon coating is contiguous with the second zone of thickness of the amorphous hydrogenated carbon coating. For example, in the embodiment of FIG. 1, first zone of thickness 122 is contiguous with second zone of thickness 124.

However, in other embodiments, one or more additional zones of thickness of the amorphous hydrogenated carbon coating are disposed between the first zone of thickness of the amorphous hydrogenated carbon coating and the second zone of thickness of the amorphous hydrogenated carbon coating. For example, in various embodiments the one or more additional zones of thickness is a third zone of thickness disposed between and contiguous with the first and second zones of thickness. One such embodiment of a surface according to the disclosure is shown in schematic, cross-sectional view in FIG. 2. In this embodiment, a surface 200 for cell culture applications includes a substrate 210 having a fluoropolymer surface 212. The surface 200 further includes an amorphous hydrogenated carbon coating 220 disposed on the fluoropolymer surface 212 of the substrate 210. The amorphous hydrogenated carbon coating 220 has a first zone of thickness 222 proximal to and extending from the fluoropolymer surface 212, and a second zone of thickness 224 distal to the fluoropolymer surface 212 and at a surface 226 of the amorphous hydrogenated carbon coating 220. The amorphous hydrogenated carbon coating 220 has a higher total concentration of oxygen and nitrogen in the second zone of thickness 224 than in the first zone of thickness 222. Notably, in this embodiment, a third zone of thickness 228 is disposed between and contiguous with the first and second zones of thickness 222 and 224.

The one or more zones of thickness disposed between the first and second zones can have a variety of compositions. Notably, in some embodiments, the third zone of thickness can include oxygen and/or nitrogen, and the amorphous hydrogenated carbon coating can have a higher total concentration of oxygen and nitrogen in the third zone of thickness than in the first zone of thickness.

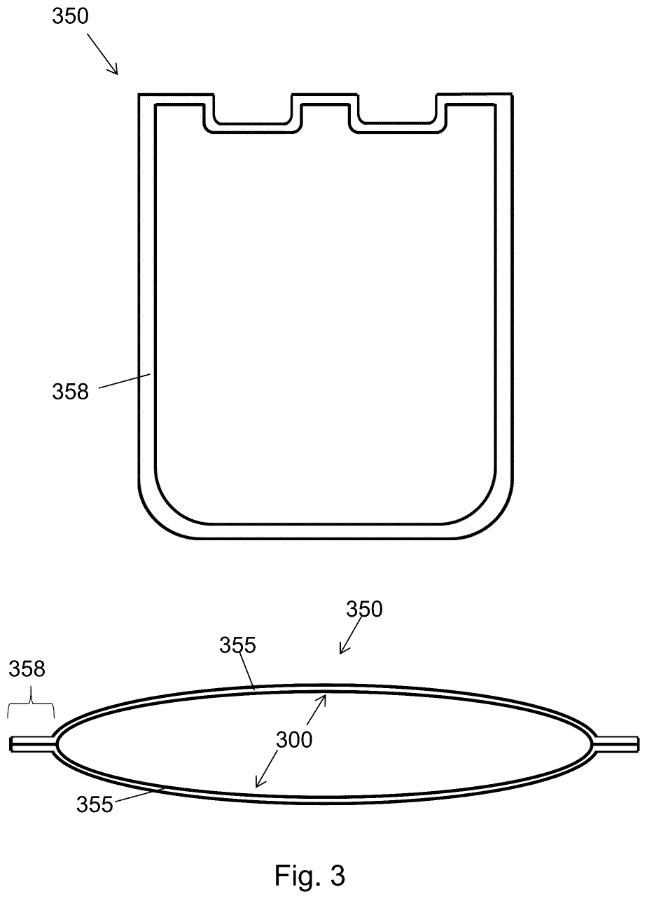

The surfaces described herein can be used in a variety of cell culture systems. For example, in various embodiments as otherwise described herein, the surface is an inner surface of a cell culture container. A variety of cell culture containers, especially those made from fluoropolymeric materials, can be adapted with the surfaces of the disclosure, e.g., by deposition of the amorphous hydrogenated carbon coating thereon. For example, in various desirable embodiments, the cell culture container is a cell culture bag. One such embodiment is shown in schematic top view (top of figure) and in schematic cross-sectional view (bottom of figure) in FIG. 3. Here, cell culture bag 350 is formed by two sheets of polymer film 355 having a fluoropolymer surface, laminated together at edges 358 thereof. The surface 300 as described herein is provided at inner surfaces of the cell culture bag. The coating can, in some embodiments, be absent at the laminated edges, so as not to interfere with lamination. Many of the fluoropolymers described above can be adapted for use in such bags. Various fluoropolymer materials described above, including the fluoropolymer silicone laminates, can be adapted for use in such cell culture bags. Of course, other cell culture containers can be adapted for use with the surfaces described herein, e.g., in the form of flasks, vials, tubes and tubings.

Another aspect of the disclosure is a method for making a surface suitable for cell culture applications, e.g., a surface as described herein. The method includes depositing on a fluoropolymer surface of a substrate an amorphous hydrogenated carbon coating. The deposition includes depositing via chemical vapor deposition (e.g., PECVD) a first thickness of the amorphous hydrogenated carbon coating in a first zone thereof using a combination of a hydrocarbon gas (e.g., ethylene or propylene) and one or more oxygen- and/or nitrogen-containing plasma-reactive gases (e.g., CO₂ and/or NH₃) at a first ratio of hydrocarbon gas to oxygen- and/or nitrogen-containing plasma-reactive gases. The first zone is proximal to and extends from the fluoropolymer surface. The deposition further includes depositing via chemical vapor deposition (e.g., PECVD) a second thickness of the amorphous hydrogenated carbon coating in a second zone thereof using a combination of a hydrocarbon gas (e.g., ethylene or propylene) and one or more oxygen- and/or nitrogen-containing plasma-reactive gases (e.g., CO₂ and/or NH₃) at a second ratio of hydrocarbon gas to oxygen- and/or nitrogen-containing plasma-reactive gases that is lower than the first ratio of hydrocarbon gas to oxygen- and/or nitrogen-containing plasma-reactive gases (e.g., CO₂ and/or NH₃). The second zone is distal to the fluoropolymer surface and at a surface of the amorphous hydrogenated carbon coating. Notably, the deposited amorphous hydrogenated carbon coating has a higher total concentration of oxygen and nitrogen in the second zone thereof than in the first zone thereof.

The first and second thicknesses of the amorphous hydrogenated carbon coating can be deposited with no layers in between, e.g., to provide a surface in which the first zone is contiguous with the second zone as described with respect to FIG. 1. In other embodiments, one or more thicknesses of amorphous hydrogenated carbon coating can be deposited in between the first and second thicknesses, as described above with respect to FIG. 2. For example, in various embodiments as otherwise described herein, the method can further include, before the deposition of the second thickness of the amorphous hydrogenated carbon coating in the second zone thereof, depositing via chemical vapor deposition (e.g., PECVD) a third thickness of the amorphous hydrogenated carbon coating in a third zone thereof using a combination of a hydrocarbon gas (e.g., ethylene or propylene) one or more oxygen- and/or nitrogen-containing plasma-reactive gases (e.g., CO₂ and/or NH₃) at a third ratio of hydrocarbon gas to oxygen- and/or nitrogen-containing plasma-reactive gases that is lower than the first ratio of hydrocarbon gas to oxygen- and/or nitrogen-containing plasma-reactive gases. In this embodiment, after the deposition of the second thickness of the amorphous hydrogenated carbon coating in the second zone thereof as described herein, the third zone is between and contiguous with the first and second zones of the amorphous hydrogenated carbon coating. Notably, the amorphous hydrogenated carbon coating can have a higher total concentration of oxygen and nitrogen in the third zone than in the first zone of the amorphous hydrogenated carbon coating.

The person of ordinary skill in the art can adapt conventional techniques for depositing amorphous hydrogenated carbon for use in the methods described herein. Plasma enhanced chemical vapor deposition (PECVD) is an especially useful technique. As used herein, a hydrocarbon gas or a plasma-reactive gas need not be a gas under ambient conditions; rather, it need be a gas under the chemical vapor deposition conditions used.

A variety of hydrocarbon gases can be used. For example, chemical vapor deposition of an alkene such as propylene or ethylene or 1,3-butadiene can be used to provide amorphous hydrogenated carbon coatings. But other hydrocarbon gases can be used, such as alkynes (e.g., acetylene, methylacetylene) and alkanes (e.g., methane, ethane, propane).

Similarly, a variety of oxygen- and/or nitrogen-containing gases can be used, provided they are reactive in the plasma conditions. A variety of oxygen-containing gases, such as alcohols (e.g., methanol, ethanol), ketones (e.g., acetone), carboxylic acids (e.g., formic acid), aldehydes (e.g., formaldehyde, acetaldehyde), and ethers (e.g., dimethyl ether) can be used to provide oxygen content to a deposited material. Carbon dioxide is an especially desirable oxygen-containing gas. Oxygen gas can be used, but at higher concentrations may act as an etchant. Similarly, a variety of nitrogen-containing gases can be used to provide nitrogen content to a deposited material, such as amines (e.g., methylamine, ethylamine, allylamine), imines (e.g., methanimine), and nitriles (e.g., acetonitrile). Ammonia is an especially desirable nitrogen-containing gas. Some plasma-reactive compounds can include both oxygen and nitrogen. e.g., nitrogen oxides and amides (e.g., formamide, acetamide).

By varying the relative amounts of hydrocarbon gas, oxygen-containing gas and nitrogen-containing gas, the person of ordinary skill in the art can provide a changing concentration of oxygen and/or nitrogen between various zones of the coating to provide the various zones described herein. For example, mass ratios of hydrocarbon gas to oxygen-containing gas (e.g., ethylene:CO2) can in some embodiments be in the range of 0.5-20, and mass ratios of hydrocarbon gas to nitrogen containing gas (e.g., ethylene:NH3) can in some embodiments be in the range of 0.1-3. It can be desirable to use a higher ratio of hydrocarbon gas to oxygen-containing gas and/or a higher ratio of hydrocarbon gas to nitrogen containing gas in deposition of the first zone than in deposition of the second zone, to provide a more highly cross-linked layer in the first zone and a more highly-functionalized layer in the second zone.

It is noted that changes in oxygen and nitrogen content can be continuous; concentrations need not be uniform within any zone as described herein.

As noted above, it can be desirable for the coating to be performed on an activated fluoropolymer surface. As noted above, an activated fluoropolymer surface can improve the adhesion of the coating to the fluoropolymer surface. Accordingly, in various embodiments of the methods described herein, the fluoropolymer surface of the substrate on which the amorphous hydrogenated carbon coating is deposited is an activated fluoropolymer surface, e.g., as described above. In various embodiments, the method further includes activating the fluoropolymer surface. The fluoropolymer surface can be activated by a plasma treatment, such as by treatment with a NH₃ plasma, a CO₂ plasma, or a plasma of a combination of NH₃ and CO₂. In other embodiments, the plasma is an oxygen plasma, a nitrogen plasma, or a plasma of a combination of oxygen and nitrogen. In other embodiments, the fluoropolymer surface can be activated by a corona treatment, such as a C-treatment. In various embodiments as otherwise described herein, the activated fluoropolymer surface has a concentration of oxygen less than 5%, and a concentration of nitrogen less than 10%. In various embodiments as otherwise described herein, the activated fluoropolymer surface can have a water contact angle in a range from 60° to 120°.

Another aspect of the disclosure is a method for culturing adherent cells that includes incubating a surface as described herein with adherent cells and a growth medium. For example, the surface may be, for example, a surface of a cell culture device (e.g., a cell culture bag, a cell culture tube, a cell culture vial or a cell culture tubing).

A variety of adherent cells can be cultured using the surfaces of the disclosure. For example, in an embodiment, the cells are stem cells, e.g., mesenchymal stromal cells (hMSCs). In another embodiment, the cells are dendritic cells, e.g., monocyte-derived dendritic cells. The person of ordinary skill in the art will identify other cell types suitable for growth.

The person or ordinary skill in the art will identify suitable growth media for use in culturing adherent cells. In various desirable embodiments as otherwise described herein, the growth medium is a basal medium. In various embodiments, the growth medium includes a buffer (e.g., sodium bicarbonate), glutamine, and a growth factor. In various desirable embodiments as otherwise described herein, the growth medium has a pH in a range of 7.2-7.4.

The person of ordinary skill in the art will identify appropriate incubation conditions for use in the growth methods described herein. For example, in various desirable embodiments as otherwise described herein, an incubation temperature for culturing the adherent cells is in the range of 35-39° C. (e.g., 37° C.).

Notably, the surfaces and containers described herein can be sterilized before use in cell culture. For example, sterilization can be performed by autoclaving. It is a significant advantage that various surfaces described herein can be sterilized by autoclaving before use, while still retaining significant function.

### EXAMPLES

The following Examples serve to illustrate various preferred embodiments and aspects of the disclosure and are not to be construed as limiting the scope thereof.

### Example 1. Activation of a Fluoropolymer Surface Using a CO₂ Plasma

This Example describes the activation of a fluoropolymer surface using a CO₂ plasma. First, the fluoropolymer surface, here, a fluorinated ethylene propylene (FEP) polymer surface, is cleaned using 70% ethanol in an ultrasonic bath sonicator for 30 min., followed by rinsing the fluoropolymer surface with reverse osmosis water and drying the fluoropolymer surface under vacuum overnight. The cleaned fluoropolymer surface is disposed on an electrode and loaded into a PECVD reactor. The PECVD reactor is generally described in M. Buddhadasa & P.-L. Girard-Lauriault, “Plasma co-polymerisation of ethylene, 1,3-butadiene and ammonia mixtures: Amine content and water stability,” Thin Solid Films, 591, 76-85 (2015), which is hereby incorporated herein by reference in its entirety. The surface is treated with CO₂ plasma for 45 s at a pressure of 13.3 Pa, a radio frequency power of 60 W, a CO₂ flow rate of approximately 20 standard cubic centimeters per minute (sccm). The activated fluoropolymer surface is sealed in a low oxygen transmission bag inside a glovebox under argon and stored −40° C. While in this Example, the activated fluoropolymer surface was removed from the PECVD reactor and stored, the person of ordinary skill in the art will appreciate that the activation and subsequent coating deposition can be performed in sequence in the PECVD reactor.

### Example 2. Activation of a Fluoropolymer Surface Using an Ammonia (NH₃) Plasma

This Example describes the activation (e.g., treatment) of a fluoropolymer surface using a NH₃ plasma. First, the fluoropolymer surface, here, a fluorinated ethylene propylene (FEP) polymer surface, is cleaned using 70% ethanol in an ultrasonic bath sonicator for 30 min., followed by rinsing the fluoropolymer surface with reverse osmosis water and drying the fluoropolymer surface under vacuum overnight. The cleaned fluoropolymer surface is disposed on an electrode and loaded into a PECVD reactor. The surface is treated with NH₃ plasma for 45 s at a pressure of 13.3 Pa, a radio frequency power of 60 W, a NH₃ flow rate of approximately 15 sccm. The activated fluoropolymer surface is sealed in a low oxygen transmission bag inside a glovebox under argon and stored −40° C. While in this Example, the activated fluoropolymer surface was removed from the PECVD reactor and stored, the person of ordinary skill in the art will appreciate that the activation and subsequent coating deposition can be performed in sequence in the PECVD reactor.

### Example 3. Deposition of a Coating Having an Oxygen-Containing Cross-Linked Layer onto an Activated Fluoropolymer Surface

This Example describes the deposition of an amorphous hydrogenated carbon coating having an oxygen-containing cross-linked layer onto an activated fluoropolymer surface as described herein. Particularly, an oxygen-rich cross-linked layer is deposited onto an activated fluoropolymer surface via PECVD under a pressure of 80 Pa and an RF power of 20 W for 10 min with mixture of ethylene (C₂H₄) at a flow rate of approximately 5 sccm and CO₂ with a flow rate of approximately 40 sccm. The coated fluoropolymer surface is sealed in a low oxygen transmission bag inside a glovebox under argon and stored at −40° C.

### Example 4. Deposition of a Coating Having an Oxygen-Containing Functional Layer onto an Activated Fluoropolymer Surface

This Example describes the deposition of a coating having an oxygen-containing functional layer onto an activated fluoropolymer surface as described herein. Particularly, an oxygen-containing functional layer is deposited onto an activated fluoropolymer surface via PECVD under a pressure of 80 Pa and an RF power of 20 W for 10 min with mixture of ethylene (C₂H₄) at a flow rate of approximately 5-2.5 sccm (ramped linearly over the 10 minute time) and CO₂ with a flow rate of approximately 40 sccm. The coated fluoropolymer surface is sealed in a low oxygen transmission bag inside a glovebox under argon and stored at −40° C.

### Example 5. Deposition of a Coating Having an Oxygen-Containing Cross-Linked Layer and an Oxygen-Containing Functional Layer onto an Activated Fluoropolymer Surface

This Example describes the deposition of a coating having an oxygen-containing cross-linked layer and an oxygen-containing functional layer onto an activated fluoropolymer surface as described herein. First, an oxygen-containing cross-linked layer is deposited onto an activated fluoropolymer surface via PECVD under a pressure of 80 Pa and an RF power of 20 W for 5 min with mixture of ethylene (C₂H₄) at a flow rate of approximately 5 sccm and CO₂ with a flow rate of approximately 40 sccm. Then, an oxygen-containing functional layer is deposited onto the oxygen-rich cross-linked layer via PECVD under a pressure of 80 Pa and an RF power of 20 W for 1.5 min with mixture of ethylene (C₂H₄) at a flow rate ranging from approximately 5 to 2.5 sccm (ramped linearly over the 1.5 minutes) and CO₂ with a flow rate of approximately 40 sccm. The coated fluoropolymer surface is sealed in a low oxygen transmission bag inside a glovebox under argon and stored at −40° C.

### Example 6. Deposition of a Coating Having a Nitrogen- and Oxygen-Containing Cross-Linked Layer onto an Activated Fluoropolymer Surface

This Example describes the deposition of a coating having a nitrogen- and oxygen-containing cross-linked layer onto an activated fluoropolymer surface as described herein. Particularly, a nitrogen- and oxygen-containing cross-linked layer is deposited onto an activated fluoropolymer surface via PECVD under a pressure of 80 Pa and an RF power of 20 W for 15 min with mixture of ethylene (C₂H₄) at a flow rate of 20 sccm, CO₂ at a flow rate of approximately 20 sccm and NH₃ at a flow rate of approximately 5 sccm. The coated fluoropolymer surface is sealed in a low oxygen transmission bag inside a glovebox under argon and stored at −40° C.

### Example 7. Deposition of a Coating Having a Nitrogen-Containing Functional Layer onto an Activated Fluoropolymer Surface

This Example describes the deposition of a coating having a nitrogen-containing functional layer onto an activated fluoropolymer surface as described herein. Particularly, a nitrogen-containing functional layer is deposited onto the activated fluoropolymer surface via PECVD under a pressure of 80 Pa and an RF power of 20 W for 90 seconds with mixture of ethylene (C₂H₄) at a flow rate ranging from 20 to 10 sccm (ramping linearly over the 90 seconds) and NH₃ at a flow rate of approximately 15 sccm. The coated fluoropolymer surface is sealed in a low oxygen transmission bag inside a glovebox under argon and stored at −40° C.

### Example 8. Deposition of a Coating Having a Nitrogen- and Oxygen-Containing Cross-Linked Layer and a Nitrogen-Containing Functional Layer onto an Activated Fluoropolymer Surface

This Example describes the deposition of a coating having an oxygen-rich cross-linked layer and a nitrogen-rich functional layer onto an activated fluoropolymer surface as described herein. First, an oxygen-containing cross-linked layer is deposited onto an activated fluoropolymer surface via PECVD under a pressure of 80 Pa and an RF power of 20 W for 3 min with mixture of ethylene (C₂H₄) at a flow rate of approximately 20 sccm, CO₂ at a flow rate of approximately 20 sccm and NH₃ at a flow rate of approximately 5 sccm. Then, a nitrogen-containing functional layer is deposited onto the nitrogen- and oxygen-containing cross-linked layer via PECVD under a pressure of 80 Pa and an RF power of 20 W for 1.5 min with mixture of ethylene (C₂H₄) at a flow rate ranging from approximately 20 to approximately 10 sccm (ramping linearly over the 1.5 min) and NH₃ at a flow rate of approximately 15 sccm. The coated fluoropolymer surface is sealed in a low oxygen transmission bag inside a glovebox under argon and stored at −40° C.

### Example 9. Elemental Analyses of Oxygen and of Nitrogen on the Surface Described in Examples 1-8

Elemental analyses were performed of the surfaces of Examples 1-8 by X-ray photoelectron spectroscopy (XPS). For XPS analysis, a Thermo Scientific K-Alpha instrument equipped with a monochromatic Al Kα radiation X-ray source was used. The coated surfaces were mounted on the vacuum transfer module in the glovebox and transported to the XPS instrument to avoid any exposure to air. Survey spectra were obtained using a 160 eV pass energy and a 200 ms dwell time on a 400 UM spot size with the Flood Gun on. Also, high-resolution spectra were obtained using 20 eV pass energy and a 200 ms dwell time. The surface composition was determined using the Thermo Fisher Scientific Advantage Software (Version: 5.9922).

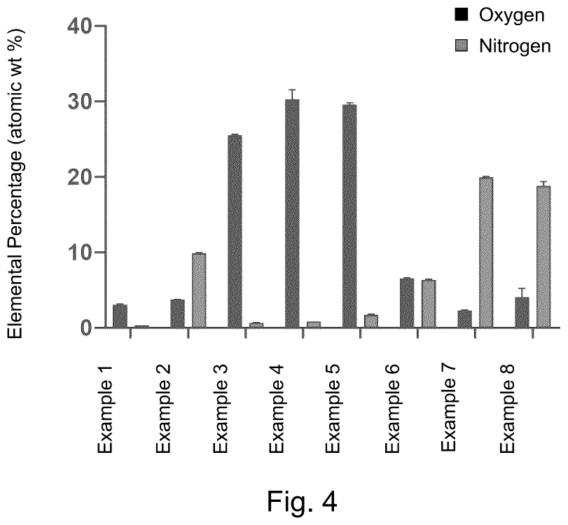

FIG. 4 is a diagram showing an elemental percentage of oxygen and of nitrogen on the surface described in Examples 1-8. It is noted that the data demonstrates a minor degree of cross-contamination between examples.

The O/C and N/C atomic ratios of the coatings of Examples 3-8 are shown in Table 1, below. Some nitrogen is present in the Example 6 coatings due to contamination.

TABLE 1

| Sample | O/C | N/C |
|---|---|---|
| Example 3 | 0.35 | 0.01 |
| Example 4 | 0.46 | 0.01 |
| Example 5 | 0.43 | 0.02 |
| Example 6 | 0.08 | 0.07 |
| Example 7 | 0.03 | 0.26 |
| Example 8 | 0.05 | 0.24 |

### Example 10. Water Contact Angles of the Surface Described in Examples 1-8

Water contact angles of the surfaces of Examples 1-8 were measured.

The water contact angles are measured using a goniometer (Future Digital Scientific Corp.) connected to a video camera system and computer software (SCA 2.0). Contact angles were measured using the sessile drop method (3 μL Milli-Q water droplets) at room temperature. The average values from 5-point measurements were calculated and reported

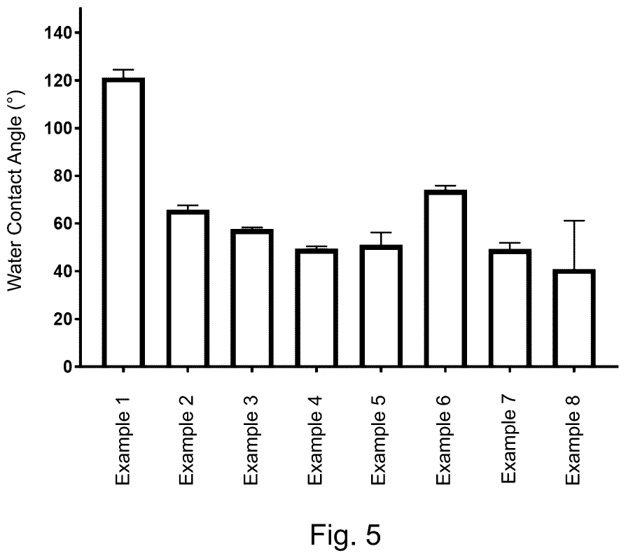

FIG. 5 is a diagram showing a water contact angle of the surface described in Examples 1-8, respectively.

### Example 11. Coating Thicknesses of Coatings Described in Examples 3-8

The thicknesses of coatings of Examples 3-8 were measured using a Dektak XT (Veeco Sloan Technology) profilometer. The FEP waviness prevented measurement of plasma polymer thicknesses below 1 μm. Therefore, the coatings were deposited on silicon wafers for the profilometry experiments. A mask covering the silicon wafers was used to create a step between the treated and untreated surfaces during plasma coating. The thickness was measured using a 12.5 μm radius stylus with a force of 3 mg. Each measurement was obtained with a resolution of 0.1 μm/point over the step region at room temperature.

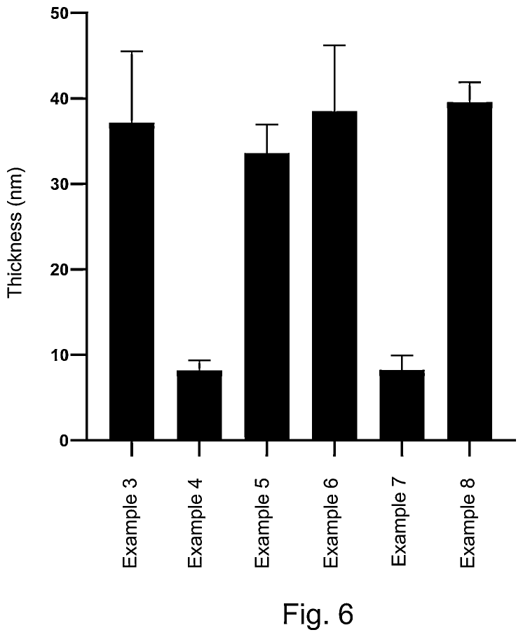

FIG. 6 is a diagram showing an exemplary thickness of a coating disposed on the surface described in Examples 3-8, respectively.

### Example 12. Effects of Sterilization on the Surfaces Described in Examples 5 and 8

This Example describes the effect of sterilization via autoclave on the surface described in Examples 5 and 8. The effects were determined by the change of an elemental percentage of oxygen and/or of nitrogen on the surface upon sterilization, and by the change of a thickness of the coating disposed on the surface upon sterilization.

Regarding the surface described in Example 5, the elemental percentage of oxygen on an unsterilized surface is determined to be approximately 29.59 atomic wt %, and the elemental percentage of oxygen on a sterilized surface is determined to reduce to approximately 25.24 atomic wt %. Hence, the approximate 14.7 atomic wt % of change in the elemental percentage of oxygen on the surface can be considered as a range for maintaining the stability of the coating on the surface.

Regarding the surface described in Example 8, the elemental percentage of nitrogen on an unsterilized surface is determined to be approximately 18.79 atomic wt %, and the elemental percentage of nitrogen on a sterilized surface is determined to reduce to approximately 13.97 atomic wt %. Hence, the approximate 25.7 atomic wt % of change in the elemental percentage of nitrogen on the surface can be considered as a range for maintaining the stability of the coating on the surface.

The thickness of the coating disposed on the surfaces tends to increase after surface sterilization. Without intending to be bound by theory, the inventors surmise that this can be due to the thermal expansion and swelling of the fluoropolymer layer at the surface. For example, the thickness of the coating on a sterilized surface as described in Example 5 is determined to increase approximately 59.7% compared to an unsterilized surface. Further, the thickness of the coating on a sterilized surface as described in Example 8 is determined to increase approximately 1.1% compared to an unsterilized surface.

### Example 13. Cell Adhesion and Proliferation of Human Mesenchymal Stromal Cells (hMSCs) on the Coated Surface Described in Examples 5 and 8, Respectively

This Example describes cell adhesion and proliferation of hMSCs on the coated surface described in Examples 5 and 8, respectively. To compare, this Example also describes cell adhesion and proliferation of hMSCs on an un-activated (e.g., untreated) fluoropolymer surface, an activated (e.g., treated) fluoropolymer surface, and commercially available tissue culture polystyrene (TCPS) surfaces (e.g., Sarstedt, red and yellow) under similar experimental conditions.

Cell adhesion and proliferation can be determined by a cell seeding density (cells/cm²) of the cells adhered on the surface. For the cell culture studies, custom wells with various FEP surfaces were prepared by mounting the FEP films onto the bottom of a CultureWell™ 8-well removable chamber slide (Gracebio) under sterile conditions. Human bone marrow-derived mesenchymal stromal cells (hMSCs, Poietics™, Lonza) were cultured using the StemMACS™ MSC expansion media XF kit (StemMACS™, Miltenyi Biotech). Frozen cells were thawed and seeded into TCPS T-75 flasks (Sarstedt®, red cap) at a density of 5,000 to 6,000 cells/cm² with a medium change at day 3. When the cells reached confluency, they were washed twice in Dulbecco's phosphate-buffered saline (DPBS) prior to removal from the surface with TrypLE (Thermo Fisher Scientific). The cells were seeded at 5000 cells/cm² onto the FEP (untreated), FEP (treated), O-GPPC, N-hyb-GPPC, TCPS (Sarstedt®, Red) and TCPS (Sarstedt®, Yellow) surfaces. The seeded cells were incubated at 37° C. and 5% CO2. The expanded cells on day 1 and day 3 were fixed, permeabilized, and stained. DAPI was used to stain the nucleus of the adhered cells. Fluorescent images were acquired using an Olympus IX81 microscope (10× objective) from 21 positions within each well. The DAPI-stained adherent cells were analyzed and counted using ImageJ.

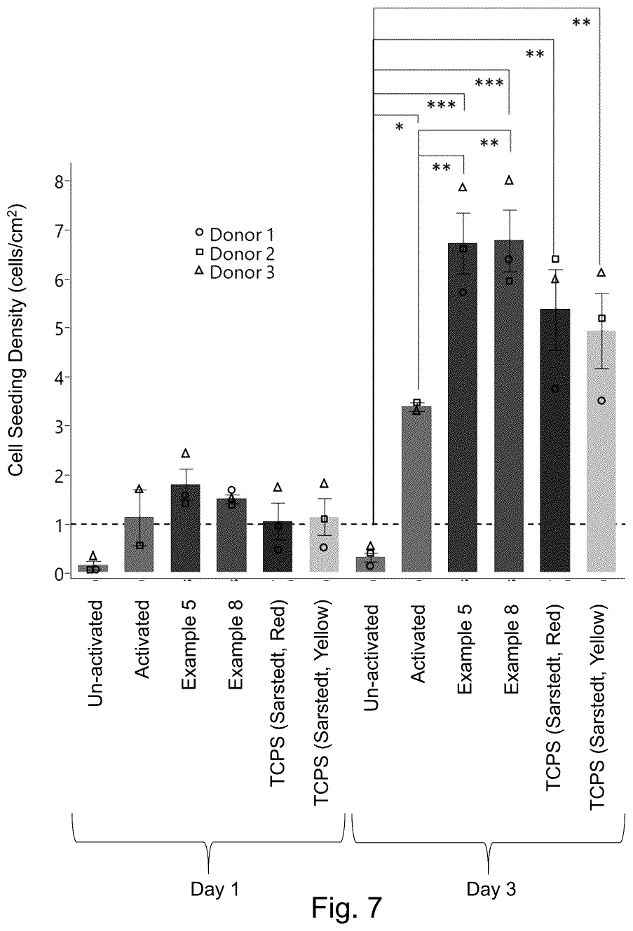

FIG. 7 is a diagram showing cell adhesion and proliferation of hMSCs on the coated surface described in Examples 5 and 8, respectively, in comparison to that on an un-activated fluoropolymer surface, an activated fluoropolymer surface, and commercially available TCPS surfaces. In general, FIG. 7 shows that hMSCs tend to adhere better on both of the coated surfaces as compared to the un-activated fluoropolymer surface, the activated fluoropolymer surface, and the commercially available TCPS surfaces under similar experimental conditions.

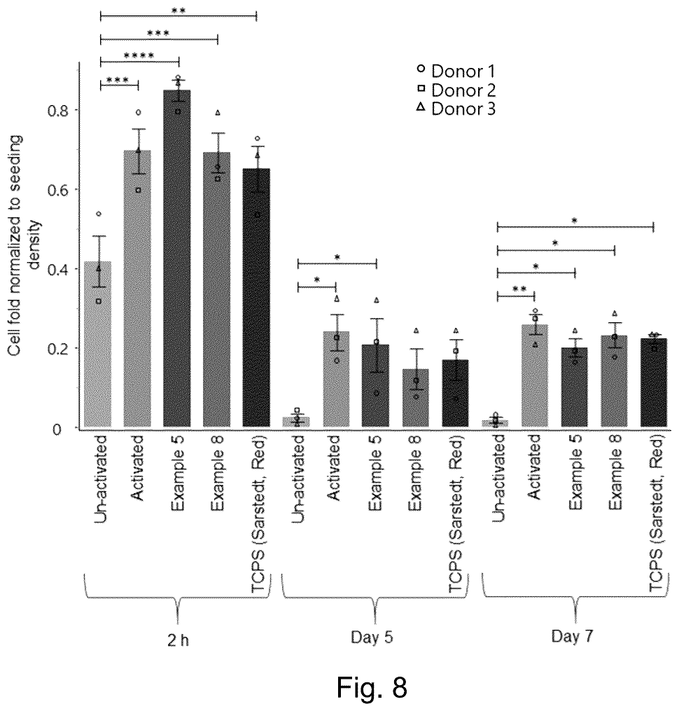

Specifically, FIG. 7 shows that in Day 1, the cell seeding densities of hMSCs on the coated surfaces are higher than those on the other tested surfaces (i.e., the un-activated fluoropolymer surface, the activated fluoropolymer surface, and the TCPS surfaces). FIG. 8 also shows that the cell seeding densities of hMSCs on the coated surfaces in Day 3 are more than three times higher than those on the same surfaces in Day 1, and that the cell seeding densities of the cells on the coated surfaces remain higher than those on the other tested surfaces in Day 3. Thus, FIG. 7 indicates that the surface chemistry of a cell culture surface, e.g., whether the surface is activated or un-activated, and/or whether the surface is coated or uncoated, can significantly influence the cell adhesion and proliferation of hMSCs on the surface, with the coated surfaces tend to significantly promote cell adhesion and proliferation under similar experimental conditions.

### Example 14. Cell Adhesion and Proliferation of Monocyte-Derived Dendritic Cells on the Coated Surface Described in Examples 5 and 8, Respectively

This Example describes cell adhesion and proliferation of monocyte-derived dendritic cells on the coated surface described in Examples 5 and 8, respectively. To compare, this Example also describes cell adhesion and proliferation of monocyte-derived dendritic cells on an un-activated (e.g., untreated) fluoropolymer surface, an activated (e.g., treated) fluoropolymer surface, and a commercially available TCPS surface under similar experimental conditions.

Isolation of primary human monocytes from fresh whole blood: Primary human monocytes were obtained through blood donation from healthy donors. Fresh blood was collected at the McGill University Health Center and transported to the Stem Cell Bioprocessing Laboratory for subsequent processing. Blood was first diluted at 1:1 ratio in DPBS supplemented with 2% human serum albumin (HSA, Sigma, Cat #A9080), after which diluted blood was gently layered on top of Histopaque®-1077 (Sigma, Cat #10771) in SepMate™-50 tubes (STEMCELL™ Technologies, Cat #85450) and exposed to a density gradient centrifugation at 1200×g. The centrifuged blood was composed of multiple layers. The layer of the buffy coat containing peripheral blood mononuclear cells (PBMCs) was collected into new 50 mL conical tubes (Fisher, Cat #1443222). The tubes were centrifuged at 300×g for 8 min, after which the supernatant was aspirated and the cell pellet consisting of the desired PBMCs was resuspended in residual volume. Cells from a single donor were pooled into one tube and washed twice by topping up the volume to 50 mL with DPBS supplemented with 2% HSA and centrifuged at 300×g for 8 min. The supernatant was aspirated, and the cell pellet was resuspended in CryoStor® CS10 (STEMCELL™ Technologies, Cat #07959) to a cell concentration of 0.5-50×106 cells/ml. The vials were kept in the −80° C. freezer for 24 h, then transferred into liquid nitrogen after 24 h for long-term storage.

Enrichment of monocytes from frozen PBMC: On the day of the experiment, frozen PBMCs were thawed in a 37° C. water bath, then diluted at a 1:1 ratio in Plasma-lyte A (ThermoFisher, Cat #NC1531549) with 10% heat-inactivated cord plasma. The content in the cryovial was transferred into a 15 ml conical tube, then centrifuged at 300×g for 5 min to remove DMSO in the CryoStor® medium, which is toxic to cells. The cells were then resuspended at a concentration of 100 million cells/mL in DPBS supplemented with 2% HSA and 1 mM EDTA (ThermoFisher, Cat #15575020), then followed by a positive selection of monocytes with EasySep™ Human CD14 Positive Selection Kit II (STEMCELL™ Technologies). Based on the manufacturer's protocol, monocytes were enriched by the immunomagnetic cell sorting by labeling CD14+ cells with a cocktail of tetrameric antibodies targeting CD14 antigen and dextran-coated magnetic particles. The isolated monocytes were suspended in ImmunoCult™-ACF Dendritic Cell Medium (STEMCELL™ Technologies) with the differentiation factors (GM-CSF 50 ng/ml; IL-4 35 ng/ml) at a concentration of 1.0×106 cells/mL.

Cell adhesion, differentiation and maturation of monocyte-derived dendritic cells: For the cell culture studies, custom wells with various FEP surfaces were prepared by mounting the FEP films onto the bottom of a CultureWell™ 8-well removable chamber slide (Gracebio) in sterile conditions.

Day 0: Cells were seeded at a density of 1,500 cells/mm2 onto the FEP (untreated), FEP (treated), O-GPPC, N-hyb-GPPC and TCPS (Sarstedt®, Red) surfaces in the ImmunoCult™-ACF Dendritic Cell Medium (STEMCELL™ Technologies) with the differentiation factors (GM-CSF 50 ng/ml; IL-4 35 ng/ml). The seeded cells were incubated at 37° C. and 5% CO2 and allowed to adhere for 2 hours.

Quantification of adherent cells: Following incubation, non-adherent cells from each well were aspirated, and the adhered cells were rinsed off the surface by washing them three times with DPBS. Adherent cells were stained by incubating in the Ethidium homodimer, and Hoechst mixture was diluted 1:400 in DPBS for 15 min. The stained cells were enumerated by acquiring fluorescent images using an Olympus IX81 inverted fluorescence microscope (10× objective) from 21 positions within each well. The stained adherent cells were analyzed and counted using ImageJ.

Day 3—Differentiation media change: Fresh Immunocult differentiation media was prepared with 2× differentiation factor (GM-CSF=50 ng/ML; IL-4=35 ng/ml). Samples were removed from the incubator and the differentiation media was added to the respective wells. The chambers were gently shaken so the media is mixed inside the wells, and the cells were again incubated at 37° C. and 5% CO₂.

Day 5—Quantification of adherent cells: Following incubation, non-adherent cells from each well were aspirated, and the adhered cells were rinsed off the surface by washing them three times with DPBS. Adherent cells were stained by incubating in the Ethidium homodimer, and Hoechst mixture was diluted 1:400 in DPBS for 15 min. The stained cells were enumerated by acquiring fluorescent images using an Olympus IX81 inverted fluorescence microscope (10× objective) from 21 positions within each well. The stained adherent cells were analyzed and counted using ImageJ.

Day 5—Maturation Media change: For the maturation of dendritic cells, fresh Immunocult maturation media was prepared with differentiation factor (GM-CSF=50 ng/ml; IL-4=35 ng/ml) and maturation factor (MPLA 2.5 μg/mL, IFN-γ 1000 U/mL). Samples were removed from the Incubator and the media containing non-adherent cells was aspirated from the wells and transferred to labeled 15 mL centrifuge tubes. To avoid the death of the adhered cells, the maturation media was added immediately to the empty wells. The aspirated media was centrifuged in the 15 mL tubes at 300×g for 5 min. The supernatant was aspirated and the cell pellet was resuspended in the required maturation media. The resuspended cell suspension was added to the respective wells, and the cells were Incubated at 37° C. and 5% CO₂.

Day 7—Quantification of adherent cells: Following incubation, non-adherent cells from each well were aspirated, and the adhered cells were rinsed off the surface by washing them three times with DPBS. Adherent cells were stained by incubating in the Ethidium homodimer, and Hoechst mixture was diluted 1:400 in DPBS for 15 min. The stained cells were enumerated by acquiring fluorescent images using an Olympus IX81 inverted fluorescence microscope (10× objective) from 21 positions within each well. The stained adherent cells were analyzed and counted using ImageJ

FIG. 8 is a diagram showing cell adhesion and proliferation of monocyte-derived dendritic cells on the coated surface described in Examples 5 and 8, respectively, in comparison to that on an un-activated fluoropolymer surface, an activated fluoropolymer surface, and a commercially available TCPS surface. In general, FIG. 8 shows that the cells tend to adhere better on both of the coated surfaces as compared to the un-activated fluoropolymer surface, and that the cell adhesions of the cells on both of the coated surfaces are comparable to those on the activated fluoropolymer surface and the commercially available TCPS surface under similar experimental conditions.

Specifically, FIG. 8 shows that at 2 h, the cell seeding density of monocyte cells on the coated surface described in Example 5 is more than double of that on the un-activated fluoropolymer surface. FIG. 8 also shows that at 2 h, the cell seeding density of monocyte cells on the coated surface described in Example 8 is about 1.5 times higher than that on the un-activated fluoropolymer surface, and is comparable to that on the activated fluoropolymer surface and the commercially available TCPS surface. Further, in either Day 5 or Day 7, the cell seeding densities of dendritic cells on all of the tested surfaces are shown lower than those at 2 h, but the cell adhesions of the cells on both of the coated surfaces are shown comparable to those on the activated fluoropolymer surface and the commercially available TCPS surface under similar experimental conditions.

Various aspects of the disclosure are further described by the following listing of enumerated embodiments, which may be combined in any combination and any number not logically or technically inconsistent.

Embodiment 1. A surface suitable for cell culture, the surface comprising:
a substrate having a fluoropolymer surface; and
an amorphous hydrogenated carbon coating disposed on the fluoropolymer surface of the substrate, the amorphous hydrogenated carbon coating having a first zone of thickness proximal to and extending from the fluoropolymer surface and a second zone of thickness distal to the fluoropolymer surface and at a surface of the amorphous hydrogenated carbon coating, the amorphous hydrogenated carbon coating having a higher total concentration of oxygen and nitrogen in the second zone of thickness than in the first zone of thickness.
   Embodiment 2. The surface according to claim 1, wherein the fluoropolymer surface is a fluorinated ethylene propylene (FEP) surface.
   Embodiment 3. The surface according to claim 1, wherein the fluoropolymer surface is a polytetrafluoroethylene (PTFE) surface, a perfluoroalkoxy (PFA) surface, an ethylene tetrafluroethylene (ETFE) surface, a polyvinylidene fluoride (PVDF) surface, a polychlorotrifluoroethylene (PCTFE) surface, an ethylene chlorotrifluoroethylene (ECTFE) surface, an ethylene fluorinated ethylene propylene (EFEP) surface, a perfluoropolyether (PFPE) surface, a modified polytetrafluoroethylene (TFM) surface, a polyvinyl fluoride surface, or a combination of any two or more thereof.
   Embodiment 4. The surface according to any of claims 1-3, wherein the fluoropolymer surface is a surface of a fluoropolymer-silicone laminate film.
   Embodiment 5. The surface according to any of claims 1-4, wherein the fluoropolymer surface is a surface of a film having an oxygen permeability of at least 1500 cc/m²-day-atm, e.g., at least 1800 cc/m²-day-atm, or in the range of 1500-20000 cc/m²-day-atm, or 1800-16000 cc/m²-day-atm.
   Embodiment 6. The surface according to any of claims 1-5, wherein the fluoropolymer surface is a surface of a film having a carbon dioxide permeability of at least 3500 cc/m²-day-atm, e.g., 4000 cc/m²-day-atm, or in the range of 3500-25000 cc/m²-day-atm, 4000-23000 cc/m²-day-atm.
   Embodiment 7. The surface according to any of claims 1-6, wherein the fluoropolymer surface is an activated fluoropolymer surface.
   Embodiment 8. The surface according to claim 7, wherein the activated fluoropolymer surface has a concentration of oxygen less than 5 atom %, and a concentration of nitrogen less than 10 atom %.
   Embodiment 9. The surface according to claim 7 or claim 8, wherein the activated fluoropolymer surface has a water contact angle in a range from 60° to 120°.
   Embodiment 10. The surface according to any of claims 7-9, wherein the fluoropolymer surface is activated by a plasma treatment.
   Embodiment 11. The surface according to claim 10, wherein the plasma is an ammonia plasma, a CO₂ plasma, or a plasma of a combination of ammonia and CO₂.
   Embodiment 12. The surface according to claim 10, wherein the plasma is an oxygen plasma, a nitrogen plasma, or a plasma of a combination of oxygen and nitrogen.
   Embodiment 13. The surface according to any of claims 7-9, wherein the fluoropolymer surface is activated by a corona treatment such as a C-treatment.
   Embodiment 14. The surface according to any of claims 1-13, wherein the amorphous hydrogenated carbon coating has a thickness in the range of 10-200 nm, e.g., 10-100 nm, or 10-75 nm, or 10-50 nm, or 15-200 nm, or 15-100 nm, or 15-75 nm, or 15-50 nm, or 20-200 nm, or 20-100 nm, or 20-75 nm, or 20-50 nm.
   Embodiment 15. The surface according to any of claims 1-14, wherein the amorphous hydrogenated carbon coating has a ratio of hydrogen to carbon in the range of at least 1, e.g., at least 1.2, or at least 1.4.
   Embodiment 16. The surface according to any of claims 1-15, wherein the first zone of thickness of the amorphous hydrogenated carbon coating has a thickness in the range of 8-190 nm, e.g., 8-100 nm, or 8-75 nm, or 8-50 nm, or 15-190 nm, or 15-100 nm, or 15-75 nm, or 15-50 nm, or 20-190 nm, or 20-100 nm, or 20-75 nm, or 20-50 nm.
   Embodiment 17. The surface according to any of claims 1-16, wherein in the first zone of thickness, the amorphous hydrogenated carbon coating has a total concentration of oxygen and nitrogen at least 3 atom % less than that a total concentration of oxygen and nitrogen in the second zone of thickness, e.g., at least 4 atom % less, or at least 5 atom % less, or at least 6 atom % less, or at least 7 atom % less.
   Embodiment 18. The surface coating according to any of claims 1-17, wherein the amorphous hydrogenated carbon coating has, in the first zone of thickness, a total concentration of oxygen and nitrogen of at least 5 atom %, e.g., at least 10 atom %, or at least 15 atom %.
   Embodiment 19. The surface according to any of claims 1-18, wherein the second zone of thickness of the amorphous hydrogenated carbon coating has a thickness in the range of 2-100 nm, e.g., 2-50 nm, or 2-35 nm, or 2-20 nm, or 2-10 nm, or 5-100 nm, or 5-50 nm, or 5-35 nm, or 5-20 nm, or 5-10 nm, or 10-100 nm, or 10-50 nm, or 10-35 nm, or 10-20 nm, or 20-100 nm, or 20-50 nm.
   Embodiment 20. The surface according to any of claims 1-19, wherein in the second zone of thickness, the amorphous hydrogenated carbon coating has a total concentration of oxygen and nitrogen of at least 10 atom %, e.g., at least 15 atom %, or at least 20 atom %.
   Embodiment 21. The surface according to any of claims 1-20, wherein in the first zone of thickness, the amorphous hydrogenated carbon coating comprises oxygen.
   Embodiment 22. The surface according to claim 21, wherein the amorphous hydrogenated carbon coating in the first zone of thickness thereof is made up of at least 90 atom %, e.g., at least 95 atom % carbon and oxygen.
   Embodiment 23. The surface according to claim 21 or claim 22, wherein in the second zone of thickness, the amorphous hydrogenated carbon coating comprises oxygen.
   Embodiment 24. The surface according to any of claims 21-23, wherein the amorphous hydrogenated carbon coating in the second zone of thickness thereof is made up of at least 90 atom %, e.g., at least 95 atom % carbon and oxygen.
   Embodiment 25. The surface according to claim 21 or claim 22, wherein in the second zone of thickness, the amorphous hydrogenated carbon coating comprises nitrogen.
   Embodiment 26. The surface according to any of claims 21, 22 and 25, wherein the amorphous hydrogenated carbon coating in the second zone of thickness thereof is made up of at least 90 atom %, e.g., at least 95 atom % carbon and nitrogen.
   Embodiment 27. The surface according to claim 21 or claim 22, wherein in the second zone of thickness, the amorphous hydrogenated carbon coating comprises oxygen and nitrogen.
   Embodiment 28. The surface according to any of claims 21, 22 and 27, wherein the amorphous hydrogenated carbon coating in the second zone of thickness thereof is made up of at least 90 atom %, e.g., at least 95 atom % carbon, oxygen and nitrogen.
   Embodiment 29. The surface according to any of claims 1-20, wherein in the first zone of thickness, the amorphous hydrogenated carbon coating comprises oxygen and nitrogen.
   Embodiment 30. The surface according to claim 29, wherein the amorphous hydrogenated carbon coating in the first zone of thickness thereof is made up of at least 90 atom %, e.g., at least 95 atom % carbon, oxygen and nitrogen.
   Embodiment 31. The surface according to claim 29 or claim 30, wherein in the second zone of thickness, the amorphous hydrogenated carbon coating comprises oxygen.
   Embodiment 32. The surface according to any of claims 29-31, wherein at least 90 atom %, e.g., at least 95 atom % of the amorphous hydrogenated carbon coating in the second zone of thickness thereof is made up of carbon and oxygen.
   Embodiment 33. The surface according to claim 29 or claim 30, wherein in the second zone of thickness, the amorphous hydrogenated carbon coating comprises nitrogen.
   Embodiment 34. The surface according to any of claims 29, 30 and 33, wherein the amorphous hydrogenated carbon coating in the second zone of thickness thereof is made up of at least 90 atom %, e.g., at least 95 atom % carbon and nitrogen.
   Embodiment 35. The surface according to claim 29 or claim 30, wherein in the second zone of thickness, the amorphous hydrogenated carbon coating comprises oxygen and nitrogen.
   Embodiment 36. The surface according to any of claims 29, 30 and 35, wherein the amorphous hydrogenated carbon coating in the second zone of thickness thereof is made up of at least 90 atom %, e.g., at least 95 atom % carbon, oxygen and nitrogen.
   Embodiment 37. The surface according to any of claims 1-20, wherein in the first zone of thickness, the amorphous hydrogenated carbon coating comprises nitrogen.
   Embodiment 38. The surface according to claim 37, wherein at least 90 atom %, e.g., at least 95 atom % of the amorphous hydrogenated carbon coating in the first zone of thickness thereof is made up of carbon and nitrogen.
   Embodiment 39. The surface according to claim 37 or claim 38, wherein in the second zone of thickness, the amorphous hydrogenated carbon coating comprises oxygen.
   Embodiment 40. The surface according to claims 37-39, wherein at least 90 atom %, e.g., at least 95 atom % of the amorphous hydrogenated carbon coating in the second zone of thickness thereof is made up of carbon and oxygen.
   Embodiment 41. The surface according to claim 37 or claim 38, wherein in the second zone of thickness, the amorphous hydrogenated carbon coating comprises nitrogen.
   Embodiment 42. The surface according to claims 37, 38 and 41, wherein at least 90 atom %, e.g., at least 95 atom % of the amorphous hydrogenated carbon coating in the second zone of thickness thereof is made up of carbon and nitrogen.
   Embodiment 43. The surface according to claim 37 or claim 38, wherein in the second zone of thickness, the amorphous hydrogenated carbon coating comprises oxygen and nitrogen.
   Embodiment 44. The surface according to claims 37, 38, and 43, wherein at least 90 atom %, e.g., at least 95 atom % of the amorphous hydrogenated carbon coating in the second zone of thickness thereof is made up of carbon, nitrogen and oxygen.
   Embodiment 45. The surface according to any of claims 1-44, wherein the first zone of thickness of the amorphous hydrogenated carbon coating is contiguous with the second zone of thickness of the amorphous hydrogenated carbon coating.
   Embodiment 46. The surface according to any of claims 1-44, wherein one or more additional zones of thickness of the amorphous hydrogenated carbon coating are disposed between the first zone of thickness of the amorphous hydrogenated carbon coating and the second zone of thickness of the amorphous hydrogenated carbon coating.
   Embodiment 47. The surface according to claim 46, wherein the one or more additional zones of thickness is a third zone of thickness disposed between and contiguous with the first zone of thickness of the amorphous hydrogenated carbon coating and the second zone of thickness of the amorphous hydrogenated carbon coating.
   Embodiment 48. The surface according to claim 47, wherein the second zone of thickness comprises nitrogen, and wherein the third zone of thickness comprises oxygen and/or nitrogen, and wherein the amorphous hydrogenated carbon coating has a higher total concentration of oxygen and nitrogen in the third zone of thickness than in the first zone of thickness.
   Embodiment 49. The surface according to any of claims 1-48, wherein the surface is an inner surface of a cell culture container, e.g., a cell culture bag, a cell culture flask, a cell culture vial, a cell culture tube or a cell culture tubing.
   Embodiment 50. A method for making a surface suitable for cell culture, e.g., a surface of any of claims 1-49, the method comprising:
depositing on a fluoropolymer surface of a substrate an amorphous hydrogenated carbon coating, the deposition including:
   depositing via chemical vapor deposition a first thickness of the amorphous hydrogenated carbon coating in a first zone thereof using a combination of a hydrocarbon gas and one or more oxygen- and/or nitrogen-containing plasma-reactive gases (e.g., CO₂ and/or NH₃) at a first ratio of hydrocarbon gas to oxygen- and/or nitrogen-containing plasma-reactive gases, wherein the first zone is proximal to and extending from the fluoropolymer surface; and
   depositing via chemical vapor deposition (e.g., PECVD) a second thickness of the amorphous hydrogenated carbon coating in a second zone thereof using a combination of a hydrocarbon gas (e.g., ethylene or propylene) and one or more oxygen- and/or nitrogen-containing plasma-reactive gases (e.g., CO₂ and/or NH₃) at a second ratio of hydrocarbon gas to oxygen- and/or nitrogen-containing plasma-reactive gases that is lower than the first ratio of hydrocarbon gas to oxygen- and/or nitrogen-containing plasma-reactive gases, wherein the second zone is distal to the fluoropolymer surface and at a surface of the amorphous hydrogenated carbon coating, and wherein the amorphous hydrogenated carbon coating has a higher total concentration of oxygen and nitrogen in the second zone thereof than in the first zone thereof.
      Embodiment 51. The method according to claim 50, further comprising before the deposition of the second thickness of the amorphous hydrogenated carbon coating in the second zone thereof, depositing via chemical vapor deposition (e.g., PECVD) a third thickness of the amorphous hydrogenated carbon coating in a third zone thereof using a combination of a hydrocarbon gas (e.g., ethylene or propylene) one or more oxygen- and/or nitrogen-containing plasma-reactive gases (e.g., CO₂ and/or NH₃) at a third ratio of hydrocarbon gas to oxygen- and/or nitrogen-containing plasma-reactive gases that is lower than the first ratio of hydrocarbon gas to oxygen- and/or nitrogen-containing plasma-reactive gases, wherein the third zone is between and contiguous with the first and second zones, and wherein the amorphous hydrogenated carbon coating has a higher total concentration of oxygen and nitrogen in the third zone than in the first zone thereof.
      Embodiment 52. The method according to claim 50 or claim 51, wherein the chemical vapor deposition is a plasma-enhanced chemical vapor deposition.
      Embodiment 53. The method according to any of claims 50-52, wherein the hydrocarbon gas is an alkene, e.g., propylene or ethylene.
      Embodiment 54. The method according to any of claims 50-53, wherein the fluoropolymer surface of the substrate on which the amorphous hydrogenated carbon coating is deposited is an activated fluoropolymer surface.
      Embodiment 55. The method according to claim 54, further comprising activating the fluoropolymer surface.
      Embodiment 56. The method according to claim 55, wherein the fluoropolymer surface is activated by a plasma treatment, e.g., treatment with a NH₃ plasma, a CO₂ plasma, a plasma of a combination of NH₃ and CO₂, an oxygen plasma, a nitrogen plasma, or a plasma of a combination of oxygen and nitrogen.
      Embodiment 57. The method according to claim 55, wherein the fluoropolymer surface is activated by a corona treatment, e.g., a C-treatment.
      Embodiment 58. The method according to any of claims 54-57, wherein the activated fluoropolymer surface has a concentration of oxygen less than 5 atom %, and a concentration of nitrogen less than 10 atom %.
      Embodiment 59. The method according to any of claims 54-58, wherein the activated fluoropolymer surface has a water contact angle in a range from 60° to 120°.
      Embodiment 60. A surface for cell culture (e.g., according to any of claims 1-49) made by a method according to any of claims 50-59.
      Embodiment 61. A method for culturing adherent cells comprising incubating the surface of any of claims 1-49 and 60 with adherent cells and a growth medium.
      Embodiment 62. The method according to claim 61, wherein the adherent cells are stem cells, e.g., mesenchymal stromal cells.
      Embodiment 63. The method according to claim 61, wherein the adherent cells are dendritic cells, e.g., monocyte-derived dendritic cells.
      Embodiment 64. The method according to any of claims 61-63, wherein the growth medium is a basal medium.
      Embodiment 65. The method according to any of claims 61-64, wherein the growth medium includes a buffer (e.g., sodium bicarbonate), glutamine, and a growth factor.
      Embodiment 66. The method according to any of claims 61-65, wherein the growth medium has a pH in a range of 7.2-7.4.
      Embodiment 67. The method according to any of claims 61-66, wherein an incubation temperature for culturing the adherent cells is in the range of 35-39° C. (e.g., 37° C.).
      Embodiment 68. The method according to any of claims 61-67, wherein the surface is sterilized before being incubated with the cells.
      Embodiment 68. The method according to claim 68, wherein the sterilization is performed by autoclaving.

What is claimed is:

1. A surface suitable for cell culture, the surface comprising:
a substrate having a fluoropolymer surface; and
an amorphous hydrogenated carbon coating disposed on the fluoropolymer surface of the substrate, the amorphous hydrogenated carbon coating having a first zone of thickness proximal to and extending from the fluoropolymer surface and a second zone of thickness distal to the fluoropolymer surface and at a surface of the amorphous hydrogenated carbon coating, the amorphous hydrogenated carbon coating having a higher total concentration of oxygen and nitrogen in the second zone of thickness than in the first zone of thickness.

2. The surface according to claim 1, wherein the fluoropolymer surface is a fluorinated ethylene propylene (FEP) surface.

3. The surface according to claim 1, wherein the fluoropolymer surface is a surface of a film having an oxygen permeability of at least 1500 cc/m²-day-atm and a carbon dioxide permeability of at least 3500 cc/m²-day-atm.

4. The surface according to claim 1, wherein the fluoropolymer surface is an activated fluoropolymer surface having a water contact angle in a range from 60° to 120°.

5. The surface according to claim 4, wherein the fluoropolymer surface is activated by a plasma treatment.

6. The surface according to claim 1, wherein the amorphous hydrogenated carbon coating has a thickness in the range of 10-200 nm.

7. The surface according to claim 1, wherein the amorphous hydrogenated carbon coating has a ratio of hydrogen to carbon in the range of at least 1.

8. The surface according to claim 1, wherein the first zone of thickness of the amorphous hydrogenated carbon coating has a thickness in the range of 8-190 nm.

9. The surface according to claim 1, wherein in the first zone of thickness, the amorphous hydrogenated carbon coating has a total concentration of oxygen and nitrogen at least 3 atom % less than that a total concentration of oxygen and nitrogen in the second zone of thickness.

10. The surface coating according to claim 1, wherein the amorphous hydrogenated carbon coating has, in the first zone of thickness, a total concentration of oxygen and nitrogen of at least 5 atom %, e.g., at least 10 atom %, or at least 15 atom %.

11. The surface according to claim 1, wherein the second zone of thickness of the amorphous hydrogenated carbon coating has a thickness in the range of 2-100 nm, e.g., 2-50 nm, or 2-35 nm, or 2-20 nm, or 2-10 nm, or 5-100 nm, or 5-50 nm, or 5-35 nm, or 5-20 nm, or 5-10 nm, or 10-100 nm, or 10-50 nm, or 10-35 nm, or 10-20 nm, or 20-100 nm, or 20-50 nm.

12. The surface according to claim 1, wherein in the second zone of thickness, the amorphous hydrogenated carbon coating has a total concentration of oxygen and nitrogen of at least 10 atom %, e.g., at least 15 atom %, or at least 20 atom %.

13. The surface according to claim 1, wherein a third zone of thickness is disposed between and contiguous with the first zone of thickness of the amorphous hydrogenated carbon coating and the second zone of thickness of the amorphous hydrogenated carbon coating.

14. The surface according to claim 13, wherein the second zone of thickness comprises nitrogen, and wherein the third zone of thickness comprises oxygen and/or nitrogen, and wherein the amorphous hydrogenated carbon coating has a higher total concentration of oxygen and nitrogen in the third zone of thickness than in the first zone of thickness.

15. The surface according to claim 1, wherein the surface is an inner surface of a cell culture container.

16. A method for making a surface suitable for cell culture according to claim 1, the method comprising:
depositing on a fluoropolymer surface of a substrate an amorphous hydrogenated carbon coating, the deposition including:
depositing via chemical vapor deposition a first thickness of the amorphous hydrogenated carbon coating in a first zone thereof using a combination of a hydrocarbon gas and one or more oxygen- and/or nitrogen-containing plasma-reactive gases (e.g., CO₂ and/or NH₃) at a first ratio of hydrocarbon gas to oxygen- and/or nitrogen-containing plasma-reactive gases, wherein the first zone is proximal to and extending from the fluoropolymer surface; and
depositing via chemical vapor deposition (e.g., PECVD) a second thickness of the amorphous hydrogenated carbon coating in a second zone thereof using a combination of a hydrocarbon gas (e.g., ethylene or propylene) and one or more oxygen- and/or nitrogen-containing plasma-reactive gases (e.g., CO₂ and/or NH₃) at a second ratio of hydrocarbon gas to oxygen- and/or nitrogen-containing plasma-reactive gases that is lower than the first ratio of hydrocarbon gas to oxygen- and/or nitrogen-containing plasma-reactive gases, wherein the second zone is distal to the fluoropolymer surface and at a surface of the amorphous hydrogenated carbon coating, and wherein the amorphous hydrogenated carbon coating has a higher total concentration of oxygen and nitrogen in the second zone thereof than in the first zone thereof.

17. A method for making a surface suitable for cell culture, the method comprising:
depositing on a fluoropolymer surface of a substrate an amorphous hydrogenated carbon coating, the deposition including:
depositing via chemical vapor deposition a first thickness of the amorphous hydrogenated carbon coating in a first zone thereof using a combination of a hydrocarbon gas and one or more oxygen- and/or nitrogen-containing plasma-reactive gases (e.g., CO₂ and/or NH₃) at a first ratio of hydrocarbon gas to oxygen- and/or nitrogen-containing plasma-reactive gases, wherein the first zone is proximal to and extending from the fluoropolymer surface; and
depositing via chemical vapor deposition (e.g., PECVD) a second thickness of the amorphous hydrogenated carbon coating in a second zone thereof using a combination of a hydrocarbon gas (e.g., ethylene or propylene) and one or more oxygen- and/or nitrogen-containing plasma-reactive gases (e.g., CO₂ and/or NH₃) at a second ratio of hydrocarbon gas to oxygen- and/or nitrogen-containing plasma-reactive gases that is lower than the first ratio of hydrocarbon gas to oxygen- and/or nitrogen-containing plasma-reactive gases, wherein the second zone is distal to the fluoropolymer surface and at a surface of the amorphous hydrogenated carbon coating, and wherein the amorphous hydrogenated carbon coating has a higher total concentration of oxygen and nitrogen in the second zone thereof than in the first zone thereof.

18. The method according to claim 17, wherein the hydrocarbon gas is an alkene

19. A surface for cell culture made by a method according to claim 17.

20. A method for culturing adherent cells comprising incubating the surface of claim 1 with adherent cells and a growth medium.
